(19) United States
(12) Plant Patent Application Publication
(10) Pub. No.: US 2007/0143901 P1
(43) Pub. Date: Jun. 21, 2007

(54) Chrysanthemum plant named Mylos

(75) Inventor: Mark Roland Boeder, The Hague (NL)

Correspondence Address: STEPTOE & JOHNSON LLP, 1330 CONNECTICUT AVENUE, N.W., WASHINGTON, DC, 20036, US

(73) Assignee: Chrysanthemum Breeders Association N.V., Rijsenhout (NL)

(21) Appl. No.: 11/304,552

(22) Filed: Dec. 16, 2005

(51) Int. Cl. A01H 5/00

(52) U.S. Cl. PLT/286

(57) ABSTRACT

A chrysanthemum plant named ‘Mylos’ characterized by its medium sized blooms with yellow ray florets and prolific branching; natural season flower date September 13-17; blooming for a period of 5 weeks.

### BACKGROUND OF THE INVENTION

‘Mylos’ is a product of a breeding and selection program for outdoor pot mums (garden mums) which had the objective of creating new chrysanthemum cultivars with a decorative type flower, a natural season flower date around September 13-17; blooming for a period of 5 weeks. The new plant of the present invention comprises a new and distinct cultivar of chrysanthemum plant. ‘Mylos’ is a seedling resulting from a crossing program, set up by a previous breeder, and which records are unknown to the inventor. The new and distinct cultivar was discovered and selected as a flowering plant by Mark Roland Boeder on a cultivated field in Rijsenhout, The Netherlands in 2001. The first act of asexual production of ‘Mylos’ was accomplished when vegetative cuttings were taken from the initial selection in 2001 and propagated further in a controlled environment in Rijsenhout, The Netherlands. The new cultivar has been found to retain its distinctive characteristics through successive propagations.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

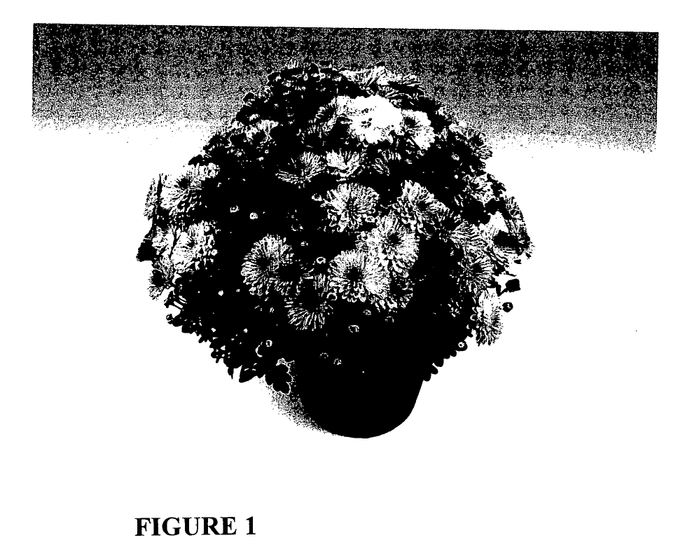

FIG. 2 shows the various stages of bloom of the new cultivar.

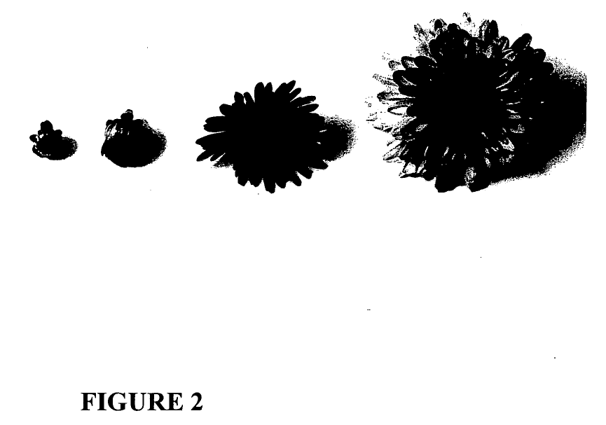

FIG. 3 shows the various stages of foliage of the new cultivar.

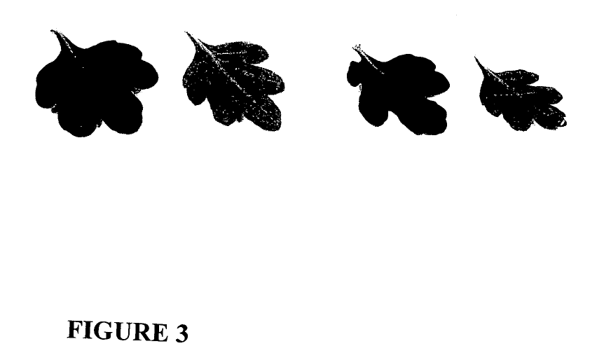

### DESCRIPTION OF THE INVENTION

This new variety of chrysanthemum is of the botanical classification Chrysanthemum morifolium L. The observations and measurements were gathered from plants grown out door in Rijsenhout, The Netherlands under natural day length and temperature and planted in week 24 in 2004. The plants were pinched once, just after planting. The natural blooming date of this crop was September 13-17 (week 38). The average height of the plants was 30 cm. No growth retardants were used. No tests were done on disease or insect resistance or susceptibility. No tests were done on cold or drought tolerance. This new variety produces medium sized blooms with yellow ray florets blooming for a period of 5 weeks.

From the cultivars known to inventor the most similar existing cultivar in comparison to ‘Mylos’ is ‘Cronus’ (U.S. Plant Pat. No. 14,563). When ‘Cronus’ and ‘Mylos’ are being compared the following difference is noticed: The difference of ‘Cronus’ and ‘Mylos’ are (1) Plant height. And (2) Color center of immature bloom. (1) The plants of ‘Cronus’ are shorter in size than those of ‘Mylos’. (2) The flower center of ‘Cronus’ is yellow in color throughout the whole blooming stage, while that of ‘Mylos’ is orange in the immature flower.

The following is a description of the plant and characteristics that distinguish ‘Mylos’ as a new and distinct variety. The color designations are taken from the plant itself. Accordingly, any discrepancies between the color designations and the colors depicted in the photographs are due to photographic tolerances. The color chart used in this description is: The Royal Horticultural Society Colour Chart, edition 1995.

### Table 1. Botanical Description of Chrysanthemum Plant ‘Mylos’

Bud:
   Size.—Small; cross-section 0.7 cm, height 0.5 cm.
   Outside color.—Yellow-green 145D.
   Involucral bracts.—2 rows, length 7 mm, width 3 mm.
   Involucral bracts among disc-florets.—Not present.
   Involucral bracts color.—Green 138A.
Bloom:
   Type.—Double.
   Height.—1.5 cm.
   Size.—Medium.
   Fully expanded.—6 cm.
   Peduncle length.—6-8 cm.
   Peduncle color.—Green 138A with ribs of Green 138C.
   Number of blooms per branch.—Approx. 5 blooms per branch.
   Performance on the plant.—5 weeks.
   Seeds.—Produced in small quantities, ovate grey-brown 199A, 1½ mm in length.
   Fragrance.—Typical chrysanthemum, slightly.
Color:
   Center of the flower.—Immature Greyed-orange 167C Mature Yellow 12A.
   Color of upper surface of the ray-florets.—Yellow 12A.
   Color of the lower surface of the ray-florets.—Yellow 10A blending to Greyed-yellow 162A at edge.
   Tonality from distance.—A garden mum with yellow flowers.
   Color of the ray-florets after aging of the plant.—Yellow 12C.
Ray florets:
   Texture.—Upper and under side smooth.
   Number.—200-210.
   Cross-section.—Flat.
   Longitudinal axis of majority.—Straight.
   Length of corolla tube.—0.6 cm.
   Ray-floret margin.—Entire.
   Ray-floret length.—2.6-2.7 cm.
   Ray-floret width.—0.7 cm.
   Ratio length/width.—Medium.
   Shape of tip.—Dentate.
Disc florets: Not present
   Receptacle shape.—Conical raised.
Reproductive organs:
   Styles.—Present in ray-florets.
   Style color.—Yellow 13A.
   Style length.—0.3 cm.
   Stigma color.—Yellow-green 144A.
   Stigma Width.—1 mm.
   Ovaries.—Enclosed in calyx.
Plant:
   Form.—Grown as a spray type potmum, outdoor mounded and round.
   Growth habit.—Spherical shape.
   Growth rate.—Moderate.
   Height.—30 cm.
   Width.—35 cm.
   Stem color.—Greyed-brown 199D.
   Stem strength.—Strong.
   Stem brittleness.—Not brittle.
   Stem anthocyanin coloration.—Absent.
   Internode length.—1-2 cm.
   Length of lateral branch.—From top to bottom 22 cm.
   Lateral branch color.—Yellow-green 148C.
   Lateral branch, attachment.—Medium strength.
   Branching (average number of lateral branches).—Prolific with 10 breaks after pinching.
   Natural season blooming date.—September 13-17.
Foliage:
   Leaf color.—Upper side Green 139 A-B Lower side Greyed-green 194A-Green 139C.
   Color midvein.—Upper side Yellow-green 147D Lower side Yellow-green 148D.
   Size.—Small; length 4.5-5 cm, width 3-4 cm.
   Quantity (number per lateral branch).—18-20.
   Shape.—Ovate.
   Texture upper side.—Glabrous.
   Texture under side.—Pubescent.
   Venation arrangement.—Palmate.
   Shape of the margin.—Serrated.
   Shape of base of sinus between lateral lobes.—Rounded.
   Margin of sinus between lateral lobes.—Parallel to diverging.
   Shape of base.—Truncate.
   Apex.—Mucronulate.
   Petiole length.—0.5 cm.
   Petiole color.—Yellow-green 147D.

TABLE 2
Differences with the comparison variety

| | “Mylos” | ‘Cronus’ |
|---|---|---|
| Plant height | 30 cm | 26 cm |
| Color flower center in | Greyed-orange 167C | Yellow 12A |
| immature stage | | |

I claim:

1. A new and distinct variety of chrysanthemum plant as described and illustrated.
